(19) United States
(12) Patent Application Publication
(10) Pub. No.: US 2014/0058148 A1
(43) Pub. Date: Feb. 27, 2014

(54) Process For Production Of Distillate Fuel

(71) Applicants: Guangci Zhou, El Cerrito, CA (US); William James Cannella, Orinda, CA (US); Stephen Harold Roby, Hercules, CA (US)

(72) Inventors: Guangci Zhou, El Cerrito, CA (US); William James Cannella, Orinda, CA (US); Stephen Harold Roby, Hercules, CA (US)

(73) Assignee: Chevron U.S.A. Inc., San Ramon, CA (US)

(21) Appl. No.: 13/594,175

(22) Filed: Aug. 24, 2012

(51) Int. Cl. C07C 1/22; C07C 5/13

(52) U.S. Cl. 585/253

(57) ABSTRACT

The present invention is directed to preparing distillate fuel having almost no oxygen and no carbon-to-carbon double bonds. The method comprises passing biodiesel and/or lipids derived from vegetable oils, algae oils, and/or animal fats over a bio-feedstock, or lipids, conversion catalyst that performs the hydrocarbon isomerization function, removes oxygen from the feedstock, cracks off the C₃ backbone, and saturates double bonds. The process is a single step process eliminating the need of a separate costly hydrotreating step while producing a renewable source distillate fuel.

### TECHNICAL FIELD

The present application relates generally to systems and methods for producing fuels from biomass, and more particularly, to systems and methods for making distillate fuels, such as diesel and jet fuels, from biomass by subjecting the biomass to catalytic reaction.

### BACKGROUND

Biofuels are of increasing interest for a number of reasons including: (1) they are a renewable resource, (2) their production is less dependent on geopolitical considerations, (3) they provide the possibility of a direct replacement of petroleum-based fuels in existing vehicles, and (4) the net greenhouse gas emissions can be substantially reduced by virtue of carbon dioxide (CO₂) uptake by biofuel precursors—particularly in the case of cellulose feedstocks.

An easily-obtainable bio-feedstock is vegetable oil, which largely comprises triglycerides and some free fatty acids. Other similar bio-feedstocks are algae oils and animal fats. However, the properties of these oils and fats generally are not sufficient for use as a direct replacement for petroleum diesel in vehicle engines, as the oils and fats can have viscosities and back-end distillation properties that are generally too high and do not burn cleanly enough, thereby leaving damaging carbon deposits on the engine. Additionally, these oils and fats gel at higher temperatures, thereby hindering their use in colder climates. These problems are mitigated when the oils and fats are blended with petroleum fuels, but still remain an impediment for long-term use in diesel engines.

Vegetable oils, algae oils, and animal fats are composed of long chain fatty acid esters of glycerol. These materials can be transesterified with methanol to produce fatty acid methyl esters (FAME), or biodiesel. Fatty acid methyl esters have some issues, though, notably poor low temperature viscometrics and poor oxidation stability, when compared to conventional hydrocarbon or petroleum-derived diesel. Fatty acid methyl esters contain a fair amount of oxygen, generally over ten percent by weight on average, and any unsaturation in the carbon chains is retained. Depending on the animal fat or vegetable oil used to make the biodiesel, the fatty acid methyl esters would contain between one and three carbon-to-carbon double bonds. However, the greater the unsaturation present in the fatty acid methyl esters, the better the low temperature properties, but poorer the oxidation stability. Poor low temperature viscometrics limit the use of fatty acid methyl esters in colder climates where the biodiesel will form wax crystals that plug fuel filters. Poor oxidation leads to the formation of insoluble gums and resins that can plug fuel filters, reducing storage stability to as little as six months as opposed to one year or longer storage stability for petroleum distillate fuel. Both of these problems can be solved by severely hydrotreating the fatty acid methyl esters or the original lipid to convert them to saturated hydrocarbons, and then isomerizing the straight chain hydrocarbons to improve their low temperature viscometrics. However, these conventional processes require a number of complex steps and the hydrotreating process is generally very costly.

Accordingly, there is a need for an improved process for high conversions of lipids and fatty acid methyl esters into acceptable distillate-compatible fuels, particularly when such a process eliminates the need for a separate hydrotreating step.

### SUMMARY

Addressing the above-described problems of preparing distillate fuels, such as diesel fuels and jet fuels, from biodiesel and/or lipids, i.e., vegetable oils, algae oils, and animal fats, provided is a process for preparing renewable distillate fuel which comprises passing biodiesel, lipid materials derived from vegetable oils and/or animal fats, or mixtures thereof over a bio-feedstock, or lipids, conversion catalyst. The process is a single step process, eliminating the need of a costly separate hydrotreating step and producing a suitable renewable distillate fuel exhibiting a cloud point of about −15° C. or below.

Among other factors, it has been found that when lipid materials such as those derived from vegetable oils, algae oils, and/or animal fats, are subjected to a bio-feedstock (or lipids) conversion catalyst, renewable distillate fuel is effectively obtained in but a single step. Specifically, it has been found that these catalysts can perform all of the reactions required to convert the lipid materials to high quality distillate fuels. These reactions include cracking off the C₃ backbone linkages of the lipids to create individual chains, removal of oxygen, saturation of double bonds, and isomerization to create branched chains. This discovery consolidates the production of renewable distillate fuel into a single step, thus cutting the capital expense and operating costs associated with a separate hydrotreater. Biodiesel can also be subjected to the single step process of the present invention, with the fatty acid methyl esters being converted in a single step to a renewable distillate fuel without the need for a separate hydrotreating step. However, by starting with the original lipid materials, one can also avoid the transesterification step of converting the lipids (vegetable oil and animal fats) to biodiesel. Overall, the present discovery allows one to prepare renewable distillate fuel in a much more simple and economical manner by consolidating the process into a single step.

### BRIEF DESCRIPTION OF THE DRAWINGS

For a more complete understanding of the exemplary embodiments of the present invention and the advantages thereof, reference is now made to the following description in conjunction with the accompanying drawings, which are briefly described as follows.

FIG. 1 is a schematic diagram of a system for producing distillate fuel, according to an exemplary embodiment.

FIG. 2 is a flowchart illustrating a method for producing distillate fuel, according to an exemplary embodiment.

### DESCRIPTION OF THE EXEMPLARY EMBODIMENTS

The present application is directed to systems and methods for producing distillate fuel, wherein such implementations comprise but a single step of passing a bio-feedstock over a bio-feedstock, or lipids, conversion catalyst to convert the feedstocks to hydrocarbon distillate fuels. The feedstock can include biodiesel, lipids of vegetable oils, algae oils, animal fats, or a mixture thereof.

Illustrative embodiments of the invention are described below. In the interest of clarity, not all features of an actual implementation are described in this specification. One of ordinary skill in the art will appreciate that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention may be better understood by reading the following description of non-limitative embodiments with reference to the attached drawings wherein like parts of each of the figures are identified by the same reference characters. The words and phrases used herein should be understood and interpreted to have a meaning consistent with the understanding of those words and phrases by those skilled in the relevant art. No special definition of a term or phrase, for example, a definition that is different from the ordinary and customary meaning as understood by those skilled in the art, is intended to be implied by consistent usage of the term or phrase herein. To the extent that a term or phrase is intended to have a special meaning, for example, a meaning other than that understood by skilled artisans, such a special definition will be expressly set forth in the specification in a definitional manner that directly and unequivocally provides the special definition for the term or phrase.

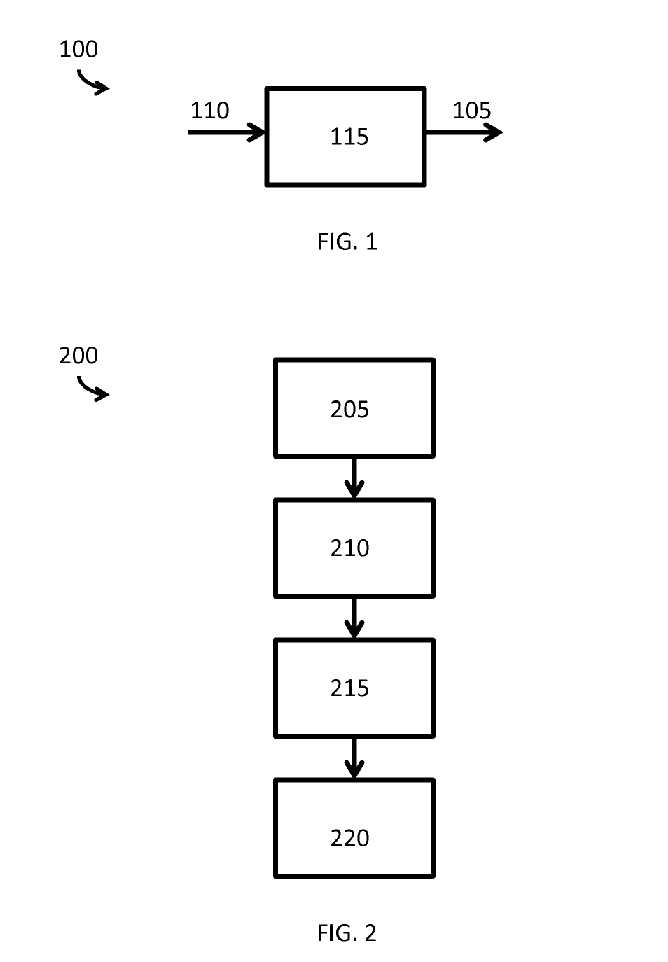

Referring to FIG. 1, a system 100 for producing a renewable hydrocarbon or distillate fuel 105 from a feed stream 110 is shown. In certain exemplary embodiments, the feed stream 110 includes fatty acid methyl esters, or biodiesel. In certain embodiments, the biodiesel includes oxygen (O₂) in an amount greater than about ten percent by weight. In certain embodiments, the biodiesel includes unsaturation in its carbon chains. In certain exemplary embodiments, the molecules of the biodiesel contain between one and three carbon-to-carbon double bonds. In certain other embodiments, the feed stream 110 includes lipids from vegetable oils, algae oils, and/or animal fats. Examples of suitable vegetable oils include, but are not limited to, crude or refined soybean, corn, coconut (including copra), palm, rapeseed, camelina, jatropha, cotton and oils. Examples of suitable animal fats include, but are not limited to, tallow, lard, butter, bacon grease and yellow grease. In certain exemplary embodiments, soybean oil is included in the feed stream 110. Generally, optimal results for producing renewable diesel fuels are obtained when using oils with C₁₆/C₁₈ carbon chains. For renewable jet fuel production, the use of oils having lower carbon numbers, for instance, approximately C₈-C₁₆, is desired, or alternatively, the heavier carbon number molecules can be selectively cracked into the jet fuel range. In yet other embodiments, the feed stream 110 includes a combination of biodiesel and lipids of vegetable oils, algae oils, and/or animal fats.

The feed stream 110 enters a catalysis unit 115 for catalytically isomerizing the feed stream 110. In certain exemplary embodiments, this step includes removal of oxygen (deoxygenation), cleavage of the C₃ glycerol backbone, hydrogenation of the double bonds, and isomerization to branched products, thus enabling a single step process. In certain embodiments, the temperature at which this step is run is generally in the range of from about 650 degrees Fahrenheit (° F.) to about 775° F. In certain exemplary embodiments, the temperature is in the range of from about 700° F. to about 775° F. By using such high temperatures, it has been found that the feed stream 110 is isomerized to the point that the recoverable diesel product, or distillate fuel 105, has a cloud point at least as low as −15° F., and in particular at least as low as −20° F., and is converted to saturated hydrocarbon chains. In certain exemplary embodiments, the feed stream 110 is subjected to the reaction step for a length of time so as to achieve a cloud point at least as low as −15° F. In certain embodiments, in a flowing reaction system, the space velocity is in a range of from about 0.25/hr to about 5.0/hr, and more preferably in the range of from about 1.0/hr to about 2.0/hr.

In certain embodiments, the feed stream 110 is in contact with a fixed stationary bed of catalyst, with a fixed fluidized bed, or with a transport bed during catalytic reaction. In one embodiment, a trickle-bed operation is employed, wherein the feed stream 110 is allowed to trickle through a stationary fixed bed, typically in the presence of hydrogen. For an illustration of the operation of such catalysts, see Miller et al., U.S. Pat. Nos. 6,204,426 and 6,723,889.

The catalytic step comprises use of a bio-feedstock, or lipids, conversion catalyst. During the reaction, the triglyceride C₃ backbone linkages are cracked, the oxygen removed, and the compounds saturated, as well as the molecules being isomerized, i.e., have more branching created. All of these functions are accomplished in but a single step using a bio-feedstock, or lipids, conversion catalyst at a temperature in the range of from about 650° F. to about 775° F. Despite the formation of water during the reactions, the catalyst has still been found to effectively catalyze isomerization in order to achieve the distillate fuel 105 with a cloud point at least as low as −15° F. Generally, the distillate fuel 105 produced contains almost no oxygen and no carbon-to-carbon double bonds. In certain embodiments, a majority of the molecules in the distillate fuel 105 includes a secondary methyl group. The resulting distillate fuel 105 would have increased energy content by removing the oxygen and improved oxidation stability by hydrogenating the double bonds. In addition, isomerization of the carbon chains improves low temperature performance.

Suitable catalysts of the present invention include, but are not limited to platinum (Pt) or palladium (Pd) on a support such as, but further not limited to SSZ-32, ZSM-23, ZSM-22, and similar such supports. In certain embodiments, the step of catalytically isomerizing the saturated, decarboxylated biofuel intermediate comprises the use of a Pt or Pd catalyst supported on an acidic support material selected from the group consisting of beta or zeolite Y molecular sieves, SiO₂, Al₂O₃, SiO2-Al₂O₃, and combinations thereof. In some such embodiments, the catalytic isomerization is carried out at a temperature between about 650° F. and about 750° F. The operating pressure is typically between about 200 pounds/square inch [gauge] (psig) to about 2000 psig, and more typically between about 200 psig to about 1000 psig. The hydrogen flow rate is typically between about 500 standard cubic feet/barrel (SCF/bbl) to about 5000 SCF/bbl.

The catalysts of the present invention include an “intermediate pore size”, or effective pore aperture, in the range of from about 5.3 angstroms (Å) to about 6.5 Å when the porous inorganic oxide is in the calcined form. Molecular sieves having pore apertures in this range tend to have unique molecular sieving characteristics. Unlike small pore zeolites such as erionite and chabazite, they will allow hydrocarbons having some branching into the molecular sieve void spaces. Unlike larger pore zeolites, such as the faujasites and mordenites, they can differentiate between n-alkanes and slightly branched alkanes, and larger branched alkanes having, for example, quaternary carbon atoms.

The effective pore size of the molecular sieves can be measured using standard adsorption techniques and hydrocarbonaceous compounds of known minimum kinetic diameters. See Breck, Zeolite Molecular Sieves. 1974 (especially Chapter 8); Anderson, et al., J. Catalysis 58,114 (1979); and Lok et al., U.S. Pat. No. 4,440,871. In performing adsorption measurements to determine pore size, standard techniques are generally employed. It is convenient to consider a particular molecule as excluded if it does not reach at least 95 percent (%) of its equilibrium adsorption value on the molecular sieve in less than about 10 minutes (p/po=0.5; 25 degrees Celsius (° C.)).

Intermediate pore size molecular sieves will typically admit molecules having kinetic diameters of about 5.3 Å to about 6.5 Å with little hindrance. Examples of such compounds (and their kinetic diameters in Å) are: n-hexane (4.3), 3-methylpentane (5.5), benzene (5.85), and toluene (5.8). Compounds having kinetic diameters of about 6 Å to about 6.5 Å can be admitted into the pores, depending on the particular sieve, but do not penetrate as quickly and in some cases are effectively excluded. Compounds having kinetic diameters in the range of about 6 Å to about 6.5 Å include: cyclohexane (6.0), 2,3-dimethylbutane (6.1), and m-xylene (6.1). Generally, compounds having kinetic diameters of greater than about 6.5 Å do not penetrate the pore apertures and thus are not absorbed into the interior of the molecular sieve lattice. Examples of such larger compounds include: o-xylene (6.8), 1,3,5-trimethylbenzene (7.5), and tributylamine (8.1).

Intermediate pore size silicoaluminophosphate molecular sieves useful in the process of the invention are SAPO-31 and SAPO-41, which are also disclosed in detail in U.S. Pat. No. 5,135,638. When combined with a Group VIII metal hydrogenation component, the lipids are converted to a mixture of substantially branched hydrocarbons.

Also useful are catalysts having intermediate pore size nonzeolitic molecular sieves, such as ZSM-22, ZSM-23 and ZSM-35, and at least one Group VIII metal. X-ray crystallography of SAPO-31, SAPO-41, ZSM-22, ZSM-23 and ZSM-35 shows these molecular sieves to have the following major and minor axes: SAPO-11, major 6.3 Å, minor 3.9 Å; (Meier, W. H., Olson, D. H., and Baerlocher, C., Atlas of Zeolite Structure Types, Elsevier, 1996), SAPO-31 and SAPO-41, believed to be slightly larger than SAPO-11, ZSM-22, major 5.5 Å, minor 4.5 Å (Kokotailo et al, Zeolites, 5, 349(85)); ZSM-23, major 5.6 Å, minor 4.5 Å; ZSM-35, major 5.4 Å, minor 4.2 (Meier, W. M. and Olsen, D. H., Atlas of Zeolite Structure Types, Butterworths, 1987).

In preparing the catalyst, the intermediate pore size molecular sieve is typically used in admixture with at least one Group VIII metal. The Group VIII metal is typically selected from the group consisting of at least one of platinum and palladium and optionally, other catalytically active metals such as molybdenum, nickel, vanadium, cobalt, tungsten, zinc and mixtures thereof. More typically, the Group VIII metal is selected from the group consisting of at least one of platinum and palladium. The amount of metal ranges from about 0.01% to about 10% by weight of the molecular sieve, preferably from about 0.2% to about 5% by weight of the molecular sieve. The techniques of introducing catalytically active metals into a molecular sieve are disclosed in the literature, and preexisting metal incorporation techniques and treatment of the molecular sieve to form an active catalyst such as ion exchange, impregnation or occlusion during sieve preparation are suitable for use in the present process. Such techniques are disclosed in U.S. Pat. Nos. 3,236,761; 3,226,339; 3,236,762; 3,620,960; 3,373,109; 4,202,996; 4,440,781 and 4,710,485.

In the present application, the term “metal” or “active metal” refers to one or more metals in the elemental state or in some form such as sulfide, oxide and mixtures thereof. Regardless of the state in which the metallic component actually exists, the concentrations are computed as if they existed in the elemental state.

It is often useful that relatively small crystal size catalyst be utilized in practicing the invention. Suitably, the average crystal size is generally no greater than about 10 microns (μm), typically no more than about 5 μm, more typically no more than about 1 μm and still more typically no more than about 0.5 μm.

The physical form of the catalyst depends on the type of catalytic reactor being employed and may be in the form of a granule or powder, and is desirably compacted into a more readily usable form (e.g., larger agglomerates), usually with a silica or alumina binder for fluidized bed reaction, or pills, prills, spheres, extrudates, or other shapes of controlled size to accord adequate catalyst-reactant contact. The catalyst may be employed either as a fluidized catalyst, or in a fixed or moving bed, and in one or more reaction stages.

The intermediate pore size molecular sieve catalyst can be manufactured into a wide variety of physical forms. The molecular sieves can be in the form of a powder, a granule, or a molded product, such as an extrudate having a particle size sufficient to pass through a 2-mesh (Tyler) screen and be retained on a 40-mesh (Tyler) screen. In cases where the catalyst is molded, such as by extrusion with a binder, the silicoaluminophosphate can be extruded before drying, or, dried or partially dried and then extruded.

The molecular sieve can be composited with other materials resistant to temperatures and other conditions employed in the catalytic process. Such matrix materials include active and inactive materials and synthetic or naturally occurring zeolites as well as inorganic materials such as clays, silica and metal oxides. The latter may be either naturally occurring or in the form of gelatinous precipitates, sols or gels including mixtures of silica and metal oxides. Inactive materials suitably serve as diluents to control the amount of conversion in the catalytic process so that products can be obtained economically without employing other means for controlling the rate of reaction. The molecular sieve may be incorporated into naturally occurring clays, e.g., bentonite and kaolin. These materials, i.e., clays, oxides, etc., function, in part, as binders for the catalyst.

Naturally occurring clays which can be composited with the molecular sieve include the montmorillonite and kaolin families, which families include the sub-bentonites, and the kaolins commonly known as Dixie, McNamee, Georgia and Florida clays or others in which the main mineral constituent is halloysite, kaolinite, diokite, nacrite or anauxite. Fibrous clays such as halloysite, sepiolite and attapulgite can also be use as supports. Such clays can be used in the raw state as originally mined or initially subjected to calcination, acid treatment or chemical modification.

In addition to the foregoing materials, the molecular sieve can be composited with porous matrix materials and mixtures of matrix materials such as silica, alumina, titania, magnesia, silica-alumina, silica-magnesia, silica-zirconia, silica-thoria, silica-beryllia, silica-titania, titania-zirconia as well as ternary compositions such as silica-alumina-thoria, silica-alumina-titania, silica-alumina-magnesia and silica-magnesia-zirconia. The matrix can be in the form of a cogel.

The catalyst used in the methods and systems of the present invention can also be composited with other zeolites such as synthetic and natural faujasites, (e.g., X and Y) erionites, and mordenites. It can also be composited with purely synthetic zeolites such as those of the ZSM series. The combination of zeolites can also be composited in a porous inorganic matrix.

FIG. 2 is a flowchart illustrating a method 200 of operation, described with respect to the system 100 of FIG. 1, according to an exemplary embodiment. In step 205, a feed stream 110 is provided. In step 210, the feed stream 110 is directed to a catalysis unit 115. In step 215, the feed stream 110 is catalytically reacted over a Group VIII metal on molecular sieve catalyst. This single catalyst performs the functions of: cracking the triglyceride C₃ backbone linkages, removing the oxygen, saturating the double bonds, and isomerizing the products without the need for a separate hydrotreating catalyst step. In step 220, the distillate fuel 105 is produced.

Relative to the process described above, however, a process which can eliminate the costly hydrotreating step and consolidate the production of renewable diesel from vegetable oils and animal fats, i.e., lipids, into a single step would be of great value. A single step process would simplify the production of renewable diesel and cuts capital expense roughly in half by eliminating the need for a hydrotreater. It would also eliminate operating costs associated with the hydrotreater.

All patents and publications referenced herein are hereby incorporated by reference to the extent not inconsistent herewith. Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. For instance, further isomerization processing adjustments can fine tune the product. Also, a narrower cut of the isodewaxed product can be isolated from a distillation column in order to obtain a desired diesel product exhibiting the properties needed. While numerous changes may be made by those skilled in the art, such changes are encompassed within the spirit of this invention as defined by the appended claims. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. The terms in the claims have their plain, ordinary meaning unless otherwise explicitly and clearly defined by the patentee.

What is claimed is:

1. A method of forming a distillate fuel product, comprising:
subjecting a feedstock to a Group VIII metal on molecular sieve catalyst, wherein the feedstock is selected from the group consisting of biodiesels, lipids derived from vegetable oils, algae oils, and animal fats, and mixtures thereof; and
catalytically reacting the feedstock over the catalyst at a temperature in the range of from 650° F. to 775° F. to produce the distillate fuel product, wherein the step of catalytically reacting the feedstock comprises catalytically cracking off its C₃ backbone linkage to create individual chains, removing molecular oxygen from the feedstock, saturating double bonds in the chains, and isomerizing the product over the catalyst.

2. The method of claim 1, wherein the temperature is in the range of from about 700° F. to 775° F.

3. The method of claim 1, wherein the reacting produces a recoverable diesel product that has a cloud point at least as low as −15° F.

4. The method of claim 3, wherein the cloud point is at least as low as −20° F.

5. The method of claim 3, wherein the cloud point is at least as low as −30° F.

6. The method of claim 1, wherein the feedstock comprises molecular oxygen in an amount of greater than about ten percent by weight.

7. The method of claim 1, wherein the biodiesel comprises unsaturation in its carbon chains.

8. The method of claim 1, wherein molecules in the biodiesel comprise between one and three carbon-to-carbon double bonds.

9. The method of claim 1, wherein the feedstock comprises soybean oil.

10. The method of claim 1, wherein the Group VIII metal on the catalyst comprises platinum or palladium.

11. The method of claim 1, wherein the Group VIII metal is present in an amount of from about 0.01% to about 10% by weight of a molecular sieve.

12. The method of claim 1, wherein the distillate fuel product contains no oxygen.

13. The method of claim 1, wherein molecules in the distillate fuel product contain no carbon-to-carbon double bonds.

14. The method of claim 1, wherein molecules in the distillate fuel product comprise a secondary methyl group.
